(19) United States
(12) Patent Application Publication
(10) Pub. No.: US 2010/0114283 A1
(43) Pub. Date: May 6, 2010

(54) IMPLANTABLE MEDICAL LEAD

(75) Inventor: Gary W. King, Fridley, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/607,482

(22) Filed: Oct. 28, 2009

Related U.S. Application Data
(60) Provisional application No. 61/110,160, filed on Oct. 31, 2008.

(51) Int. Cl. A61N 1/05

(52) U.S. Cl. 607/117; 607/116

(57) ABSTRACT

An implantable medical lead includes an elongate central body having an axis and a first extendable member pivotably moveable relative to the central body such that a portion of the extendable member is configured to move from a retracted position relative to the axis of the elongate central body to an extended position where the portion of the extendable member extends laterally beyond the central body. The lead includes a first electrode disposed on the central body and second and third electrodes disposed on the extendable member. The lead is configured such that the centers of the first, second and third electrodes are linearly arranged when the extendable member is in the extended position.

### RELATED APPLICATION

This application claims priority to U.S. Provisional Patent Application No. 61/110,160, filed on Oct. 31, 2008, which application is hereby incorporated herein by reference in its entirety to the extent that it is not inconsistent with the present disclosure.

### FIELD OF THE DISCLOSURE

This disclosure relates to implantable medical leads, and particularly to implantable medical leads that have outwardly deployable electrodes.

### BACKGROUND OF THE INVENTION

Recent efforts in the medical field continue to focus on the delivery of therapy in the form of electrical stimulation to precise locations within the human body. Therapy originates from an implanted or externally-worn source device, which may be an electrical pulse generator. Therapy is applied through one or more implanted leads that communicate with the source device and include one or more therapy delivery sites for delivering therapy to precise locations within the body. In electrical therapy systems, delivery sites take the form of one or more electrodes wired to the source device. In spinal cord simulation (SCS) techniques, for example, electrical stimulation is provided to precise locations near the human spinal cord through a lead that is usually deployed in the epidural space of the spinal cord. Such techniques have proven effective in treating or managing disease and acute and chronic pain conditions.

Percutaneous leads are small diameter leads that may be inserted into the human body, usually by passing through a Tuohy (non-coring) needle which includes a central lumen through which the lead is guided. Percutaneous leads may be inserted into the body with a minimum of trauma to surrounding tissue. On the other hand, the types of lead structure, including the electrodes, that may be incorporated into percutaneous leads is limited because the lead diameter or cross-section must be small enough to permit the lead to pass through the Tuohy needle.

Recently, the use of “paddle” leads, like Model 3586 Resume® Lead or Model 3982 SymMix® Lead of Medtronic, Inc., which offer enhanced therapy control over percutaneous leads, have become popular among clinicians. Paddle leads include a generally two-dimensional set of electrodes on one side for providing electrical therapy to excitable tissue of the body. Through selective programmed polarity (i.e., negative cathode, positive anode or off) of particular electrodes, electric current can be “steered” toward or away from particular tissue within the spinal cord or other body areas. This feature permits adjustment of the recruitment areas after the lead has been positioned in the body and therefore provides a level of adjustment for less than ideal lead placement. Additionally, the value of a transverse tripole group of electrodes has been demonstrated for spinal cord stimulation. This approach allows shielding of lateral nervous tissue with anodes, like the dorsal roots, and steering of fields in the middle under a central cathode by use of two simultaneous electrical pulses of different amplitudes.

One feature recognized in known paddle leads used for SCS is that their installation, repositioning and removal necessitates laminectomies, which are major back surgeries involving removal of part of the vertebral bone. Laminectomies are required because paddle leads have a relatively large transverse cross-sectional area compared to percutaneous leads. Thus, implantation, repositioning and removal require a sufficiently sized passage through the vertebral bone.

Another feature with paddle leads is that optimal positioning may be difficult during implant. For example, the transverse tripole leads described above work optimally if the central cathode is positioned coincident with the physiological midline of the spinal cord. Such placement can be challenging because the doctor cannot see the spinal cord through the dura during implant. Moreover, lead shifting may occur subsequent to implant, thereby affecting the efficacy of the therapy delivered from the lead.

Another feature recognized with paddle leads is that the lead position may change, sometimes solely caused by patient movement. For example, when a patient lies down, the spacing between an epidural lead and the spinal cord decreases to a large extent, so that it is often necessary to lower the amplitude of the stimulation by as much as half. It is also believed that steering effects of a tripole lead might also be affected if the cerebrospinal fluid (“CSF”) width changes dramatically, or if due to patient twisting or activity, the orientation between the lead and spinal cord changes.

### SUMMARY OF THE DISCLOSURE

Among other things, this disclosure describes a lead structure for stimulation of excitable tissue surfaces which combines the features offered by percutaneous leads with respect to minimized trauma during insertion, repositioning and removal with the advantages offered by paddle-type leads with respect to increased efficacy, ability to provide electrodes in places lateral to the axis of the lead and tailoring of treatment. Exemplary embodiments of this disclosure relate to implantable leads that have portions with electrodes that may be expanded, retracted or adjusted after implantation in the human body. Some illustrative mechanisms for accomplishing such expansion, retraction or adjustment of such leads are also described.

Exemplary embodiments of the disclosure also provide a lead structure which permits adjustment of the lead dimensions and therefore the delivery site location in situ for enhanced control of the therapy being applied to the excitable body tissues.

Exemplary embodiments of the disclosure include an implantable therapy lead having an elongate central body. These embodiments also include at least one extendable member that moves from a retracted position within, or relative to, the elongate central body to an extended position where at least a portion of the extendable member laterally extends beyond the central body. Electrodes may be disposed on the elongate central body and the extendable member. In some embodiments, an electrode on the extendable member is oriented so that it is parallel to an electrode on the central body when the extendable member is in the extended position. The lead may further include a second extendable member that extends to an extended position generally opposite to the extended position of the other extendable member.

In some embodiments, the extendable member includes a gear that engages a worm gear located at the end of a flexible actuator. The flexible actuator is capable of causing the extendable member to move from its retracted position to its extended position by rotating the flexible actuator.

In various embodiments, the extendable member has a lever portion having a flexible actuator secured to the lever portion. The flexible actuator is capable of causing the extendable member to move from its retracted position to its extended position by pulling on the flexible actuator. In some of such embodiments, the lead includes an insulating conduit for containing conductors that electrically couple electrodes of the lead to contacts of the lead. The flexible actuator may also be contained within the insulating conduit with adequate freedom of movement to allow an operator to caused the extendable member to move from its retracted position to its extended position by pulling on the flexible actuator.

In numerous embodiments, the extendable member has a lever portion having a flexible actuator secured to the lever portion. The flexible actuator is secured to the extendable member and is capable of causing the extendable member to move from its extended position to its retracted position by pulling on the flexible retractor.

In various embodiments, the extendable member includes an actuation surface and the lead further includes a movable stylet with a driver, which may be on the distal end of the stylet, that interacts with the actuation surface to cause the extendable member to move from its retracted position to its extended position by pushing on the stylet.

In some embodiments, the implantable medical lead has two rows of electrodes on the central body and at least two electrodes on the extendable member. An electrode on the extendable member is oriented so that it is aligned with a row of therapy delivery electrodes on the central body when the extendable member is in the extended position.

In many embodiments, the extendable member slides out from, or relative to, the central body in a direction generally perpendicular to the axis of the central body. In some cases, this may include a flexible actuator secured to the extendable member in such a way that pulling on the flexible actuator caused the extendable member to slide out from the central body. In other cases, a thermal deployment actuator that recovers its programmed shape may cause the extendable member to move out from the central body.

In various embodiments, an implantable medical lead includes an elongate central body having an axis and a first extendable member pivotably moveable relative to the central body such that a portion of the extendable member is configured to move from a retracted position relative to the axis of the elongate central body to an extended position where the portion of the extendable member extends laterally beyond the central body. The lead includes a first electrode disposed on the central body and second and third electrodes disposed on the extendable member. The lead is configured such that the centers of the first, second and third electrodes are linearly arranged when the extendable member is in the extended position. The lead may also include a second extendable member with an electrode disposed thereon. The second extendable member may pivot and extend in a manner generally opposite than the first extendable member. In an extended position, the center of the electrode of the second extendable member may be linearly arranged with the centers of the first, second and third electrodes.

In some exemplary embodiments of the disclosure, a method for providing therapy to a targeted tissue of a patient includes inserting a conduit having a central lumen into a patient. The method also includes implanting a lead into the patient by passing it through the central lumen of the conduit. The lead has an elongate central body with an electrode thereon and at least one extendable member having an electrode thereon. The extendable member is capable of pivoting from a retracted position within the elongate central body to an extended position. The extendable member is then deployed to the extended position to form an electrode array that includes the electrode on the central body and the electrode on the extendable member. The proximal end of the lead is then coupled to a therapy delivery device and the therapy delivery device is operated to provide treatment therapy though the electrode array. The conduit employed in this method could be a needle, a catheter, or other conduit known in the art. The therapy delivery device may be an implantable device, and the method may further include implanting the device.

In various embodiments in accordance with the disclosure, a system for treating a patient using electrical energy includes a therapy delivery device capable of generating electrical energy. The system also includes a lead having electrical conductors capable of transmitting the electrical energy. The lead has an elongate central body and at least one extendable member that pivots from a retracted position within, or relative to, the elongate central body to an extended position where at least a portion of the extendable member extends laterally beyond the central body. Electrodes are disposed on the elongate central body and the extendable member and connected to the electrical conductors. In various embodiments, an electrode on the extendable member is oriented so that it is parallel to a therapy delivery electrode on the central body when the extendable member is in the extended position.

One or more embodiments of leads, methods or systems described herein may have one or more of the following exemplary benefits relative to existing leads, systems and methods:

1. The spacing of electrodes on an extendable member or central body can be matched to important dimensions of the tissue affected, e.g., the width of the Cerebro-Spinal Fluid (CSF) between the dura and the spinal cord.

2. As the dimensions of a lead tip are changed, the locations of the electrodes relative to the tissue affected may be advantageously altered. For example, as a paddle's width is increased the paddle will move toward the spinal cord in the semicircular dorsal part of the epidural space.

3. In cases where the bones or fluid compartments have large widths (e.g., CSF depth at spinal level T7 or T8) or are too wide in a particular patient, the electrode span can be increased appropriately to ensure effective therapy.

4. Changes in paddle width and the accompanying medial and lateral movement of the sites can have a beneficial effect on the therapy. For example, the ability to stimulate only the medial dorsal columns versus the more lateral dorsal roots may provide enhanced therapeutic results.

5. As the patient ages, their pathological condition changes, their degree of fibrosis or scar tissue changes, or the effects of the therapy change, adjustments of the extendable member might restore or maintain the benefit.

6. Because of the ability to adjust the extendable member after implantation, it may be possible to optimize the benefits and minimize undesirable side effects.

7. By changing the position of the extendable member, it may be possible to avoid surgery to replace or reposition the lead.

8. By changing the position of the extendable member, it may be possible to position the electrodes optimally relative to important physiological locations, e.g., the physiological midline of nervous tissue.

9. It may be possible to minimize the use of energy by optimizing efficiency of therapy delivery through adjustment of the extendable member. This may increase battery life, a key concern for implantable devices.

10. There may be minimal insertion trauma and operating room time and resources needed if it is possible to place a lead with percutaneous techniques, and then expand it in situ.

11. Repositioning of a lead with an extendable member can be done without laminectomy. Removal may also be made quicker and less traumatic.

### BRIEF DESCRIPTION OF THE DRAWINGS

The accompanying drawings which are incorporated into and form a part of the specification, illustrate several embodiments of the present disclosure and, together with the description, serve to explain the principles of the disclosure. The drawings are only for the purpose of illustrating embodiments of the disclosure and are not to be construed as limiting the disclosure.

FIG. 1 is a plan representation of an exemplary embodiment of a lead in accordance with the present, disclosure.

FIG. 2 is a plan representation of an exemplary embodiment of a lead in accordance with the present disclosure.

FIG. 3 is a plan representation of an exemplary embodiment of a lead in accordance with the present disclosure.

FIG. 4 is a plan representation of an exemplary embodiment of a lead in accordance with the present disclosure.

FIG. 5 is a plan representation of an exemplary embodiment of a lead in accordance with the present disclosure.

FIG. 6 is a plan representation of an exemplary embodiment of a lead in accordance with the present disclosure.

FIG. 7 is a plan representation of an exemplary embodiment of a lead in accordance with the present disclosure.

FIG. 8 is a plan representation of an exemplary embodiment of a lead in accordance with the present disclosure.

FIG. 9 is a plan representation of an exemplary embodiment of a lead in accordance with the present disclosure.

FIG. 10 is a plan representation of an exemplary embodiment of a lead in accordance with the present disclosure.

FIG. 11 is a plan representation of an exemplary embodiment of a lead in accordance with the present disclosure.

FIG. 12 is a plan representation of an exemplary embodiment of a lead in accordance with the present disclosure.

FIG. 13 is a plan representation of an exemplary embodiment of a lead in accordance with the present disclosure.

The drawings presented herein are schematic and are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise. As used herein, “have”, “having”, “include”, “including”, “comprise”, “comprising” or the like are used in their open ended sense, and generally mean “including, but not limited to.”

Any direction referred to herein, such as “top,” “bottom,” “left,” “right,” “upper,” “lower,” “above,” below,” and other directions and orientations are described herein for clarity in reference to the figures and are not to be limiting of an actual device or system or use of the device or system. Devices or systems as described herein may be used in a number of directions and orientations.

The present disclosure relates to, among other things, a lead for stimulation of excitable tissue surfaces, which leads combine the features offered by percutaneous leads with respect to minimized trauma during insertion, repositioning and removal with the advantages offered by paddle-type leads with respect to increased efficacy, ability to provide electrodes in places lateral to the axis of the lead and tailoring of treatment. Exemplary embodiments of this disclosure relate to implantable leads that have portions with electrodes that may be expanded, retracted or adjusted after implantation in the human body. Some illustrative mechanisms for accomplishing such expansion, retraction or adjustment of such leads are also described.

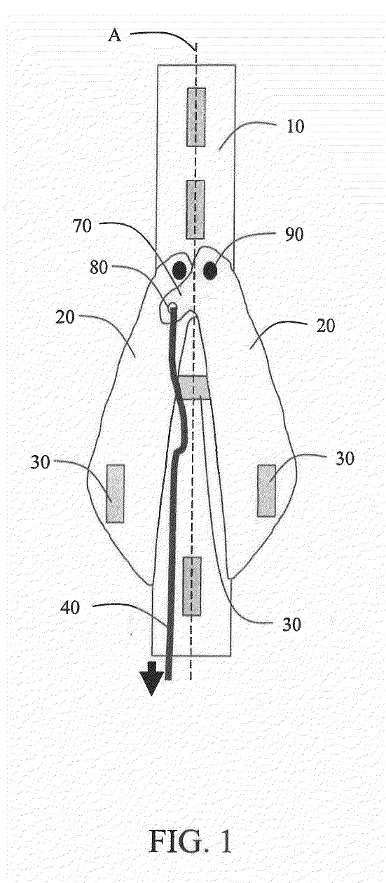

Referring now to FIG. 1, a plan representation of an embodiment of a lead is shown. FIG. 1 illustrates an embodiment of an implantable medical lead having an elongate central body 10 and extendable members 20. Electrodes 30, which may be used for therapy delivery, monitoring, sensing or the like, are located on the central body 10 and the extendable members 20. In the depicted embodiment, the lead may be implanted with the extendable members 20 in a retracted position so that they are generally contained within the central body 10 (position not shown). In some embodiments (with regard to FIG. 1 and other embodiments depicted and described herein), the extendable members 20 may be disposed exteriorly to the central body 10 when in the retracted position. Once the lead is implanted near the tissue to be stimulated, an operator can pull on a flexible actuator 40 that is attached to a lever portion 70 at attachment point 80. The pulling of the flexible actuator 40 will cause the extendable member 20 to rotate about pivot point 90, deploying the extendable member into an extended position 20 relative to the central body 10 (exemplary position shown).

In the exemplary embodiment shown in FIG. 1, the electrodes 30 on the two extendable members 20 and the central body 10 form a tri-pole stimulation array having regularity in orientation. That is, electrodes are arranged in a row across the span of the array and are oriented generally parallel to each other rather than skewed. In an exemplary embodiment, a span of 7 millimeters is achieved to provide controllable stimulation to the tissue.

In the exemplary embodiment shown in FIG. 1 and in other embodiments depicted or described herein, the central body and the lead generally may be generally round in cross-section. This increases the steerability of the lead as compared to flatter leads that may be implanted by use of a flat lam needle. Once the extendable members 20 are moved to an extended position, the benefits of a paddle-type lead may be realized from a lead that may be advantageously deployed through a round needle and with improved steerability.

The embodiment shown in FIG. 1 may be implanted in accordance with techniques know for implanting percutaneous or other leads. For example, a Tuohy needle or other catheter or conduit may be positioned near the dura of the spine. Such needles or conduits typically have an inner diameter of less than 3 millimeters, less than 2 millimeters, or about 1 to 1.5 millimeters. The lead may then be inserted through the lumen of Tuohy needle or conduit and positioned near the dura. A proximal end (not shown) of the lead body may be connected to a source device (not shown) which may be a pulse generator in the case of electrical stimulation. Although the disclosure will be described herein with reference to SCS procedures and the embodiments described in relation to electrical therapy, it will be recognized that the disclosure finds utility in applications other than SCS procedures, including, but not limited to, other applications such as peripheral nervous system (PNS) stimulation, sacral root stimulation, cortical surface stimulation, intravecular cerebral stimulation, cardiac stimulation, or monitoring of neural, cardiac, muscle, or other excitable tissue. In addition, leads in accordance with the disclosure find applicability to SCS procedures where the lead is placed in the intrathecal (subdural) space.

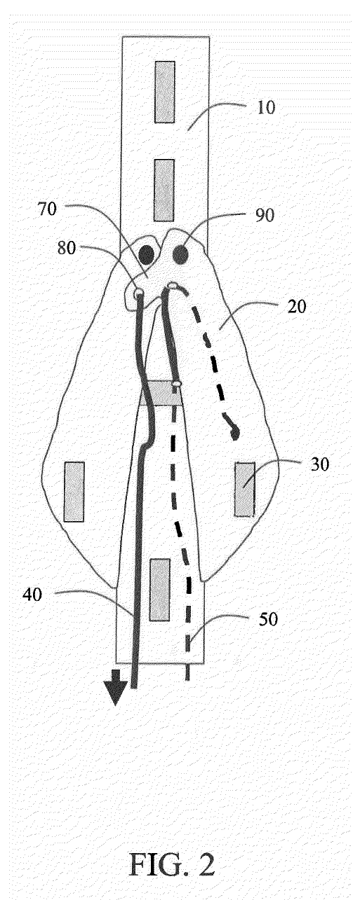

FIG. 2 is a plan representation of an embodiment of a lead that has many similar components to the embodiment of FIG. 1 as described earlier. The embodiment of FIG. 2 also shows a conductor 50 that runs through the central body 10 to the electrode 30. The conductor may be connected at its proximal end (not shown) to a source device (not shown) which may be a pulse generator in the case of electrical stimulation. Separate conductors 50 may run to each electrode 30 and be independently powered by the source device. In this way therapy delivery may be managed by selectively delivering therapy through the electrodes that will have the most impact. This is made more effective due to the breadth of the electrode array that can be created when the extendable member 20 is in deployed to its extended position and the electrodes 30 disposed on the extendable member 20 stimulate tissue at a distance from the axis A of the central body.

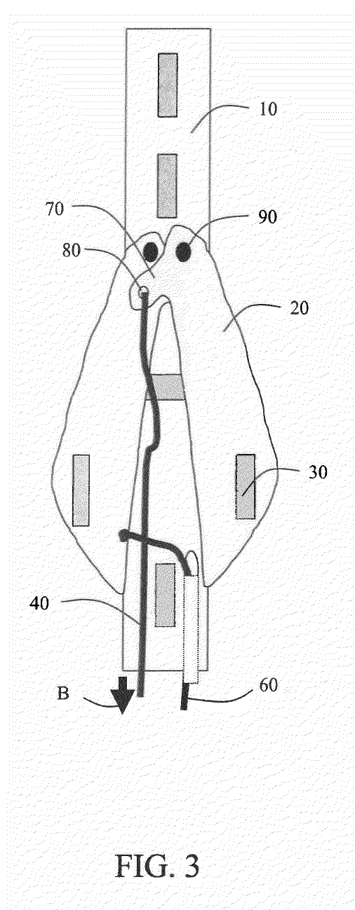

FIG. 3 is a plan representation of an embodiment of a lead that includes many elements similar to those disclosed and discussed with respect to FIGS. 1 and 2. The embodiment of FIG. 3 includes a flexible retractor 60 that is connected to the extendable member 20. In the event that the lead should be removed or repositioned, an operator can pull on flexible retractor 60 to cause the extendable members to return to a retracted position. In the event that the lead is oriented such that arrow “B” indicates the direction that the lead would need to be pulled to be removed, retraction of the extendable member 20 makes removal of the lead much easier and less traumatic for the surrounding tissue.

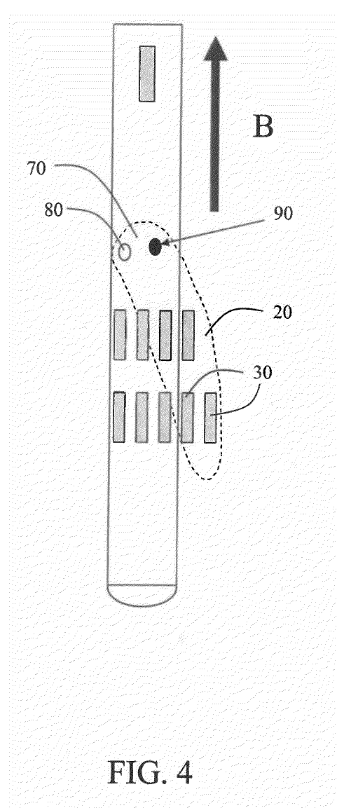

FIG. 4 is a plan representation of an embodiment of a lead that has two rows of electrodes 30, with each row containing a plurality of electrodes 30. The electrodes 30 on the extendable member 20 are oriented so that when the extendable member 20 is in its extended position (as shown) the electrodes 30 on the extendable member 20 are oriented so that they are generally parallel to the electrodes 30 on the central body 10. By being so oriented the delivery of therapy through the electrodes can be more predictably managed. In the event that the lead is oriented such that arrow “B” indicates the direction that the lead would need to be pulled to be removed, the extendable members could be biased such that removal of the lead would cause the extendable member 20 to move toward the retracted position in part due to the friction of removing the lead through the surrounding tissue.

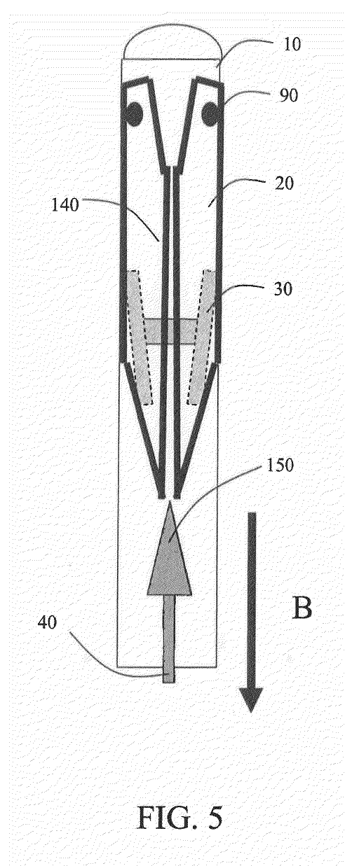

FIG. 5 is a plan representation of an embodiment of a lead that includes a central body 10 and extendable members 20 that pivots about pivot points 90. Electrodes 30 are located on the central body 10 and the extendable members 20. The extendable member 20 of this embodiment includes actuation surfaces 140. The flexible actuator 40 is a stylet that has a driver 150 on the distal end. The driver 150 interacts with the actuation surface 140 to cause the extendable member 20 to move from its retracted position (shown) to its extended position. The embodiment could be designed so that the flexible actuator remains in place as long as it is desired to keep the extendable member 20 in the extended position. Alternatively, the lead could be designed so that the extendable member 20 stays in the extended position once it is deployed.

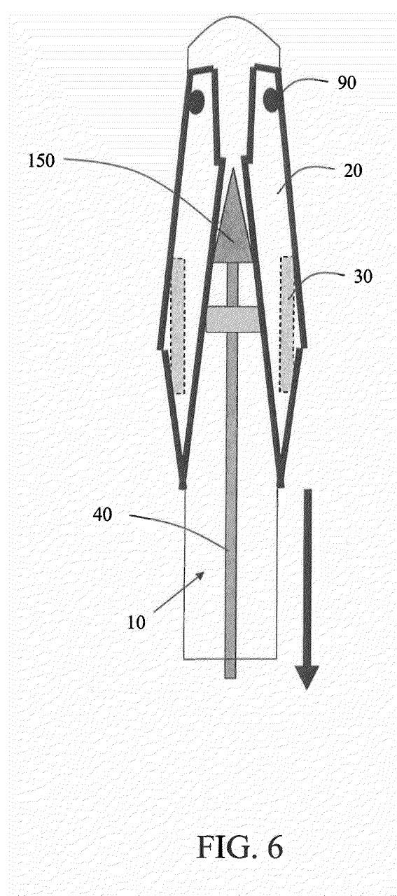

FIG. 6 is a plan representation of an embodiment of the lead shown in FIG. 5, with the extendable member 20 shown in its extended position. FIG. 6 shows how the driver 150 on the flexible actuator 40 interacts with the actuation surface 140 to drive the extendable member 20 to an extended position. As just described, the flexible actuator 40 can be left in place to hold the extendable member 20 in the extended position or the lead could be designed such that the extendable member 20 will stay in the extended position even if the flexible actuator 40 is removed.

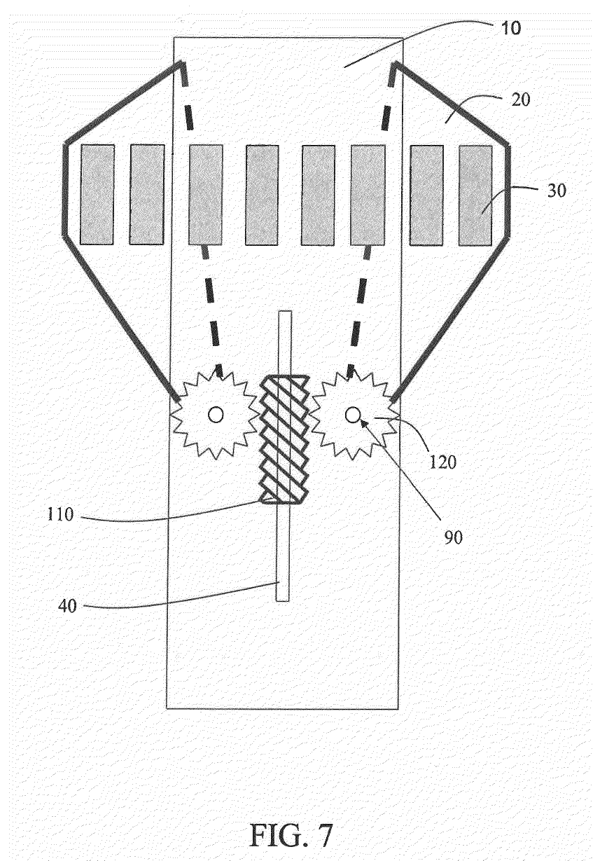

FIG. 7 is a plan representation of an embodiment of a lead that includes a central body 10, extendable member 20, electrodes 30, and flexible actuator 40. The embodiment in FIG. 7 is shown with the extendable member in an extended position. In this embodiment, the flexible actuator 40 has a worm gear 110 attached to it. The extendable member 20 has a gear 120 proximate one end that engages with the worm gear 110. Gear 120 may be a spur gear or helical gear depending primarily on the thickness of gear 120. To move the extendable member 20 to an extended position, the flexible actuator 40 is rotated about its axis, driving the worm gear 110, which drives the gear 120 to move the extendable member to the extended position. This embodiment allows for simple retraction of the extendable member 20 by simply rotating the flexible actuator 40 in the opposite direction than the direction used to extend the extendable member 20. In the embodiment shown in FIG. 7, the electrodes 30 on the extendable member 20 are parallel with the electrode on the central body 10. The extendable member could be extended to a greater or lesser extent than that represented in FIG. 7, but the position shown in FIG. 7 could be a preferred position for a variety of reasons.

The embodiment depicted in FIG. 7 also allows for precise control of the location of the extendable member 20 over the entire range of motion. A practitioner could extend or retract extendable member 20 by rotating the flexible actuator 40 until a preferred location of the extendable member 20 is achieved.

The flexible actuator 40 could be fixedly attached to the worm gear 110, or the flexible actuator 40 could have a non-circular cross section, at least at the distal end, that could be inserted into a opening on the end of the worm gear 110 of a similar size and dimension. In this way the flexible actuator 40 could be removed from the lead after the extendable member 20 has been deployed and reinserted if retraction or adjustment of the extendable member 20 position is desired.

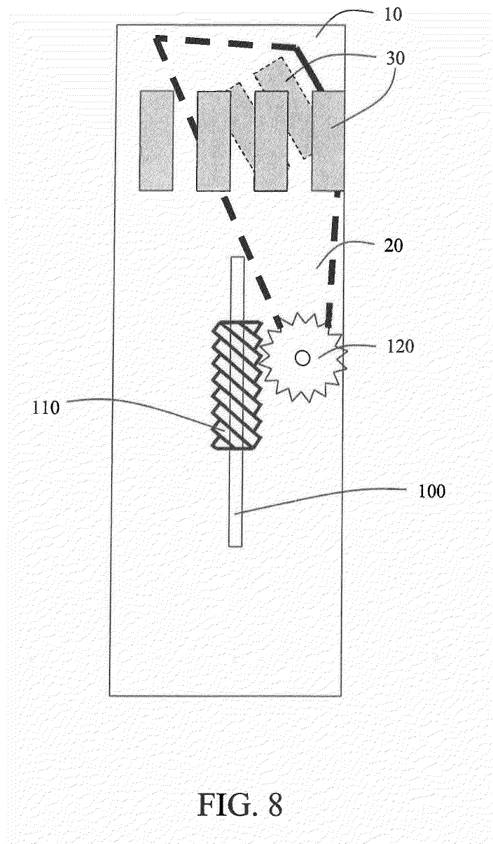

FIG. 8 is a plan representation of an embodiment of a lead that is essentially the same as the embodiment of FIG. 7 except that the extendable member 20 is shown in the retracted position. The electrodes 30 on the extendable member 20 are oriented such that when the extendable member 20 is in an extended position the electrodes on the extendable member may be parallel to those on the central body 10. As discussed above, the extendable member 20 may be extended to a greater or lesser extent depending on the treatment objectives and requirements.

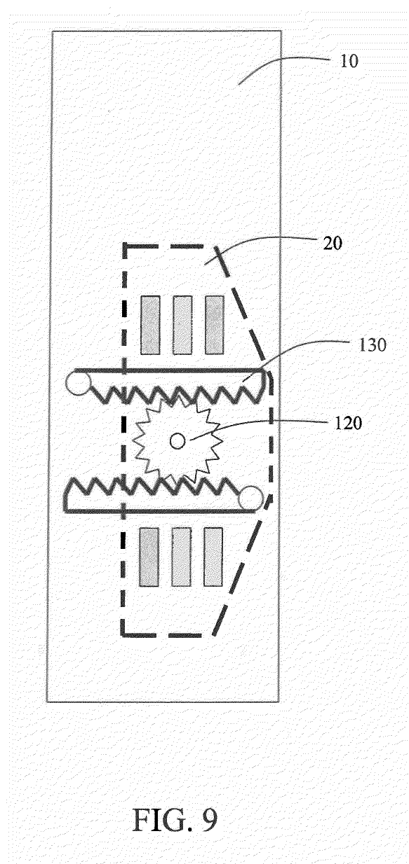

FIG. 9 is a plan representation of an embodiment of a lead in which gear 120 is oriented adjacent to a rack gear 130. The extendable member 20 is oriented to move from the retracted position (shown) to an extended position along a path that is generally perpendicular to the axis of the central body 10. Rotation of the gear 120 may be accomplished by a miniature electric motor or other means including but not limited to a flexible actuator as described herein. In this embodiment, one extendable member 20 may be located above the gear 120 and another may be located below the gear 120. In that fashion the gear 120 may cause one of the extendable members 20 to move out of the central body 10 in one direction and another extendable member 20 to move out of the central body 10 in generally the opposite direction. In some embodiments, pins located on the central body 10 may engage slots located in the extendable member 20 to keep the extendable member 20 moving smoothly from a retracted to an extended position and back. Alternatively, pins located on the extendable members 20 may engage slots on the central body 10 in much the same fashion. Other means of ensuring effective operation will occur to those of ordinary skill in the art upon reading this disclosure.

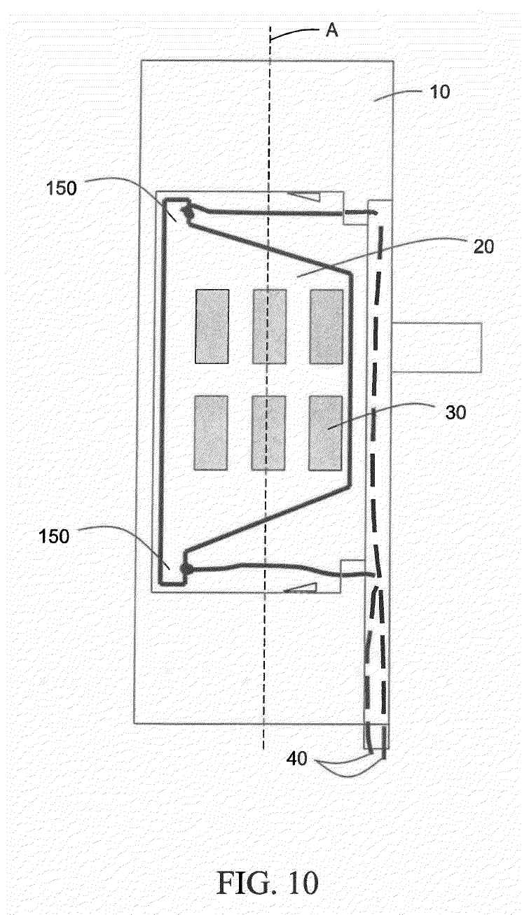

FIG. 10 is a plan representation of an embodiment of a lead in which the extendable member 20 is oriented to move from the retracted position (shown) to an extended position along a path that is generally perpendicular to the axis of the central body 10. Flexible actuators 40 are connected to tabs 150 located proximate the ends of extendable member 20. The flexible actuators are oriented such that pulling the flexible actuators drives the extendable member 20 toward extended position in a direction generally perpendicular to the axis A of the central body 10. In an alternative embodiment, a flexible actuator 20 may be connected to the extendable member 20 proximate the center of the extendable member 20. Other configurations will occur to those of skill in the art upon reading this disclosure.

As with embodiments in accordance with FIG. 9, pins located on the central body 10 may engage slots located in the extendable member 20 (or vice versa) to keep the extendable member 20 moving smoothly from a retracted to an extended position and back.

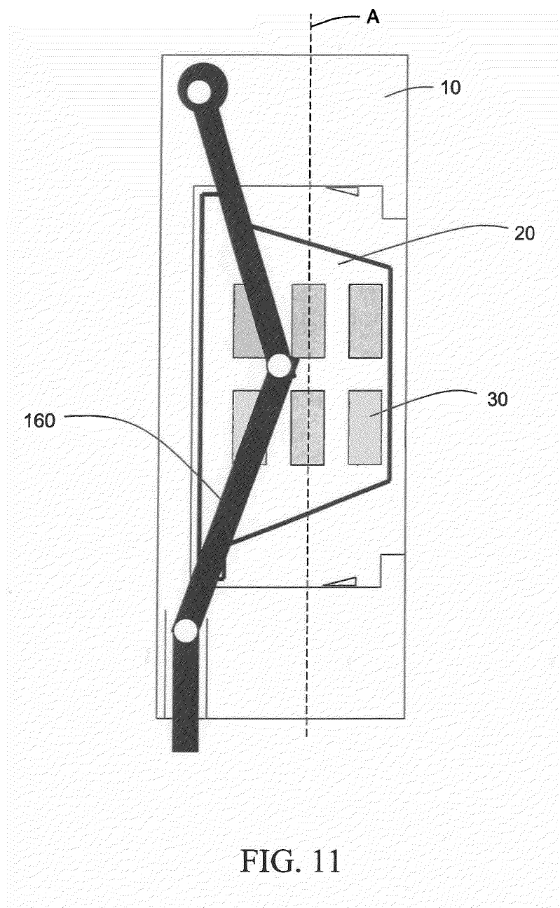

FIG. 11 is a plan representation of an embodiment of a lead that includes a central body 10 with an axis A, an extendable member 20, and electrodes 30. A thermal deployment actuator 160 includes a shape memory alloy, also known as a smart alloy or memory metal. A shape memory alloy is a metal that “remembers” its previous or original geometry. After a shape memory alloy has been deformed from its original configuration, it may regain its original geometry by heating, due to a temperature-dependent phase transformation from a low-symmetry to a highly symmetric crystallographic structure. Nitinol is but one example of such an alloy.

The thermal deployment actuator 160 may be actuated by use of an electrical signal that heats the element due to electrical resistance. The thermal deployment actuator 160 may be designed with a system of levers to provide mechanical advantage if necessary. The extendable member 20 or the central body 10 may have pins or slots to help the extendable member 20 deploy in a direction generally perpendicular to the axis of the central body.

Once in position within the patient, for example in the epidural space of the spine, a lead in accordance with embodiments of the disclosure may be deployed out of a Tuohy needle or other conduit. The extendable member 20 of the lead may be deployed in any of the manners described above to create an electrode array capable of tissue stimulation similar in effectiveness to stimulation using a more traditional paddle-type lead without the invasiveness of a laminectomy.

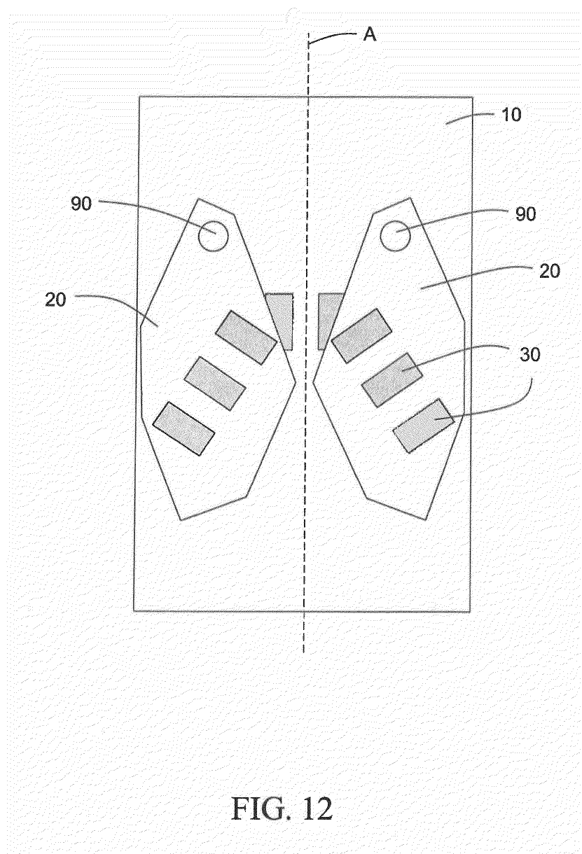

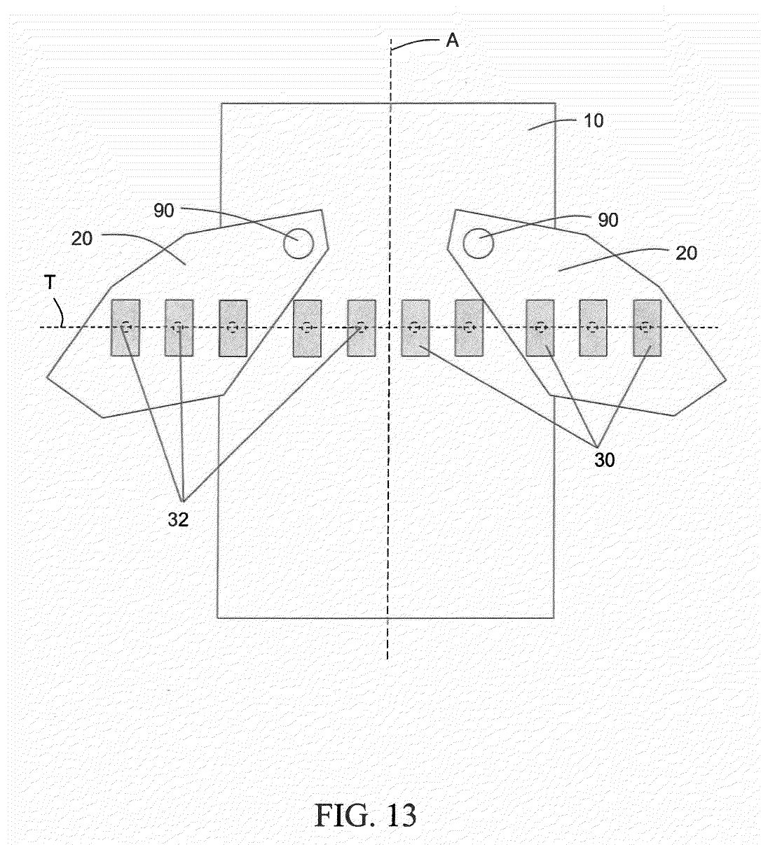

Referring now to FIGS. 12 and 13, a plan representation of a lead is shown. The lead has a central body 10 and extendable members 20 pivotably moveable relative to the central body about pivot point 90. The central body 10 has an axis A, and the extendable members 20 are configured to move from a retracted position relative to the axis A of the elongate central body 10 to an extended position where a portion of the extendable member 20 extends laterally beyond the central body 10 (FIG. 13). Electrodes 30 are disposed on the central body 10 and the extendable members 20, where more than one electrode 30 is positioned on the extendable members 20. The electrodes have geometric centers 32 (depicted as circles in the figures). As shown in the embodiment depicted in FIG. 13, the centers 32 of the electrodes 30 on the extendable members 20 are arranged linearly with the centers 32 of the electrodes 30 on the central body 10, and in the depicted embodiment are arranged along a line T transverse to the axis A of the central body. In the depicted embodiment, a plurality of electrodes 30 are disposed on the central body 10, but it will be understood that one or more electrodes may be disposed on the central body. Also, the depicted embodiment shows three electrodes disposed on the extendable member 20. In various embodiments, two or more electrodes are disposed on the extendable member, where at least the centers of a first and second electrode on the extendable member are aligned with the center of at least one electrode on the central body when the extendable members are in an extended position.

In the depicted embodiment, the electrodes 30 of the extendable members 20 are generally parallel to the electrodes 30 of the elongate body 10 when the extendable members are in an extended position (see FIG. 13). However, it will be understood the electrodes 30 need not be parallel to one another. As long as the centers 32 are generally aligned or co-linearly arranged, the electrodes should be able to provide a functional and efficacious electrode array.

Any suitable mechanism may be employed to move the extendable members 20 depicted in FIGS. 12 and 13 from a retracted position to an extended position, and optionally from an extended position to a retracted position, such as the mechanisms described above with regard to FIGS. 1-11.

Those skilled in the art will recognize that the preferred embodiments may be altered or amended without departing from the true spirit and scope of the disclosure, as defined in the accompanying claims.

What is claimed is:

1. An implantable medical lead comprising:
an elongate central body having an axis;
a first extendable member pivotably moveable relative to the central body such that a portion of the extendable member is configured to move from a retracted position relative to the axis of the elongate central body to an extended position where the portion of the extendable member extends laterally beyond the central body;
a first electrode disposed on the central body; and
second and third electrodes disposed on the extendable member,
wherein the first, second and third electrodes each have a center, and
wherein the centers of the first, second and third electrodes are co-linearly arranged when the extendable member is in the extended position.

2. The implantable medical lead of claim 1, wherein the lead is configured to be implanted through a Tuohy needle.

3. The implantable medical lead of claim 1, further comprising a second extendable member and a fourth electrode disposed on the second extendable member,
wherein the second extendable member is pivotably moveable relative to central body such that a portion of the extendable member is configured to extend relative to the axis of the central body to an extended position in a manner generally opposite the first extendable member,
wherein the fourth electrode has a center, and
wherein when the first and second extendable members are in their extended positions, the centers of the first, second, third and fourth electrodes are co-linearly arranged.

4. The implantable medical lead of claim 1, further comprising a gear that engages a worm gear located at the end of a flexible actuator, the flexible actuator being capable of causing the first extendable member to move from its retracted position to its extended position by rotating the flexible actuator.

5. The implantable medical lead of claim 1, further comprising a lever portion having a flexible actuator secured to the lever portion, the flexible actuator being capable of causing the first extendable member to move from its retracted position to its extended position by pulling on the flexible actuator.

6. The implantable medical lead of claim 5 further comprising an insulating conduit for containing conductors, wherein the flexible actuator is contained within the insulating conduit with adequate freedom of movement to allow an operator to cause the extendable member to move from its retracted position to its extended position by pulling on the flexible actuator.

7. The implantable medical lead of claim 6, further comprising a flexible retractor secured to the first extendable member, the flexible retractor being capable of causing the first extendable member to move from its extended position to its retracted position by pulling on the flexible retractor.

8. The implantable medical lead of claim 1, wherein the first extendable member includes an actuation surface configured to interact with a driver of a movable stylet to cause the first extendable member to move from its retracted position to its extended position by pushing on the stylet.

9. The implantable medical lead of claim 1, further comprising a fourth electrode on the central body, wherein the fourth electrode has a center, and wherein the centers of the first, second, third and fourth electrodes are linearly arranged when the extendable member is in the extended position.

10. The implantable medical lead of claim 9, further comprising a second extendable member and a fifth electrode disposed on the second extendable member,
wherein the second extendable member is pivotably moveable relative to the central body such that a portion of the extendable member is configured to extend laterally relative to the axis of the central body to an extended position in a manner generally opposite the first extendable member,
wherein the fifth electrode has a center, and
wherein when the first and second extendable members are in their extended positions, the centers of the first, second, third, fourth, and fifth electrodes are linearly arranged.

11. A method for providing therapy to a targeted tissue of a patient comprising:
inserting a conduit having a central lumen into a patient;
implanting a lead into the patient by passing it through the central lumen of the conduit, the lead having an elongate central body with a first electrode thereon and a first extendable member having second and third electrodes thereon, the extendable member capable of pivoting relative to the elongate central body from a retracted position to a laterally extended position;
deploying the extendable member to the extended position to form an electrode array in which the geometric centers of the first, second and third electrodes are linearly arranged;
coupling a proximal end of the lead to a therapy delivery device; and
operating the therapy delivery device to provide treatment therapy though the electrode array.

12. The method of claim 11, wherein the lead has a second extendable member capable of pivoting relative to the elongate central body from a retracted position to a laterally extended position, where a fourth electrode is disposed on the second extendable member, wherein the method further comprises deploying the second extendable member such that the geometric centers of the first, second, third and fourth electrodes are arranged linearly.

13. The method of claim 12, wherein the second extendable member is deployed in a generally opposite direction, relative to the axis of the elongate central body of the lead, from the first extendable member.

14. The method of claim 11, wherein the conduit is a needle.

15. The method of claim 11, wherein the conduit is a catheter.

16. The method of claim 11, wherein the therapy delivery device is an implantable device and the method includes the further step of implanting the device.

17. The method of claim 11, wherein the lead is implanted proximate the spinal cord and the therapy is targeted at neural tissue.

18. A system comprising:
a moveable stylet having a driver; and
an implantable medical lead comprising
(i) an elongate central body having an axis;
(ii) a first extendable member pivotably moveable relative to the central body such that a portion of the extendable member is configured to move from a retracted position relative to the axis of the elongate central body to an extended position where the portion of the extendable member extends laterally beyond the central body;
(iii) a first electrode disposed on the central body; and
(iv) second and third electrodes disposed on the extendable member,
wherein the first, second and third electrodes each have a center,
wherein the centers of the first, second and third electrodes are linearly arranged when the extendable member is in the extended position, and
wherein the first extendable member includes an actuation surface configured to interact with the driver of the movable stylet to cause the first extendable member to move from its retracted position to its extended position by pushing on the stylet when the driver is engaged with the actuation surface.

19. The system of claim 18, further comprising an insulating conduit for housing electrical conductors, wherein the stylet is moveable within the conduit.

20. The system of claim 19, wherein the actuator is housed within the insulating conduit.
